(19) United States
(12) Patent Application Publication
(10) Pub. No.: US 2022/0000367 A1
(43) Pub. Date: Jan. 6, 2022

(54) BIOMETRIC INFORMATION MEASUREMENT DEVICE, TERMINAL, AND BIOMETRIC INFORMATION MEASUREMENT SYSTEM

(71) Applicant: OMRON HEALTHCARE Co., Ltd., Kyoto (JP)

(72) Inventors: Hideie HIRATA, Kyoto (JP); Takashi KITAMURA, Kyoto (JP); Toru DENO, Kyoto (JP); Mizuki FURUTA, Kyoto (JP)

(21) Appl. No.: 17/448,149

(22) Filed: Sep. 20, 2021

Related U.S. Application Data
(63) Continuation of application No. PCT/JP2020/009149, filed on Mar. 4, 2020.

(30) Foreign Application Priority Data
Mar. 25, 2019 (JP) 2019-056855

(51) Int. Cl. A61B 5/00; A61B 5/0215; H04Q 9/00

(52) U.S. Cl. CPC A61B 5/0028; H04Q 9/00; A61B 5/0031; A61B 5/0215

(57) ABSTRACT

The present invention provides a biometric information measurement device that communicates with an external terminal by ultrasonic waves, including: a communicator for communicating information including measurement information with the external terminal; and a light projector for visualizing information on a place where the external terminal is to be placed by irradiating light.

### CROSS-REFERENCE TO RELATED APPLICATIONS

This application is the U.S. national stage application filed pursuant to 35 U.S.C. 365(c) and 120 as a continuation of International Patent Application No. PCT/JP2020/009149, filed Mar. 4, 2020, which application claims priority to Japanese Patent Application No. 2019-056855, filed Mar. 25, 2019, which applications are incorporated herein by reference in their entireties.

### TECHNICAL FIELD

The present invention relates to a biometric information measurement device, a terminal, and a biometric information measurement system.

### BACKGROUND ART

Biometric information measurement devices for measuring biometric information such as blood pressure have been developed and have become widely used. In addition, a technology for transmitting biometric information measured by a biometric information measurement device to an external terminal by using near-field wireless communication technology is disclosed (For example, Patent Document 1).

### CITATION LIST

### Patent Literature

Patent Document 1: JP 6460568 B

### SUMMARY OF INVENTION

### Technical Problem

In a case where a means of high directivity such as ultrasonic waves is employed as the medium for wireless communication between the biometric information measurement device and an external terminal (for example, a terminal such as a smartphone), in order to establish communication, it is considered desirable that the biometric information measurement device and the external terminal are in a predetermined positional relationship when performing communication.

Therefore, it is necessary to inform the user of information on a predetermined relationship. As a method of notifying the user, it is conceivable to describe a method for arranging an external terminal in an instruction manual or the like. However, in the case of such a method, it is difficult for the user to understand intuitively, and it is difficult for the user to understand. Further, a method of providing a pedestal for arranging an external terminal on the biometric information measurement device is also conceivable. However, in the case of such a method, the biometric information measurement device becomes large, which is not particularly preferable for portable devices. In other words, the present inventors have found that, in a conventional method, the user may not place the external terminal at an appropriate position with respect to the biometric information measurement device, and communication between the biometric information measurement device and the external terminal may not be established.

In one aspect, the present invention has been made in consideration of such circumstances, and an object thereof is to provide a technology for displaying an appropriate positional relationship between a biometric information measurement device and an external terminal in an easy-to-understand manner.

### Solution to Problem

The present invention adopts the following configurations in order to achieve the above-mentioned object.

In other words, a biometric information measurement device according to one aspect of the present invention is a biometric information measurement device configured to communicate with an external terminal by ultrasonic waves, the biometric information measurement device including: a communicator configured to communicate information including measurement information with the external terminal; and a light projector configured to visualize information on a place where the external terminal is to be arranged by irradiating light.

Here, the information on the place where the external terminal is to be arranged includes a place where the terminal can suitably communicate with the biometric information measurement device via ultrasonic waves. According to this configuration, since the information on the place where the external terminal is to be arranged is visualized, the user can easily understand the recommended arrangement place of the external terminal. Thus, the user can easily place the external terminal at a position with an appropriate relative relationship with respect to the biometric information measurement device. Thus, even in a case where the biometric information measurement device and the external terminal communicate by ultrasonic waves having high directivity and a short reach distance, communication is easily established.

In the biometric information measurement device according to the above-described aspect, the place irradiated with light by the light projector may include a place on a placement surface on which the host device is placed.

According to this configuration, the user can easily place the external terminal at a position with an appropriate relative relationship with respect to the biometric information measurement device.

In the biometric information measurement device according to the above-described aspect, a period of time in which light is irradiated by the light projector may include at least one of a predetermined period of time before execution of communication with the external terminal by the communicator or at least a part of period of time in which the communication is executed.

According to this configuration, when communication by the biometric information measurement device is started, the user can be prompted to place the external terminal at a place irradiated with light. Thus, the period of time in which the biometric information measurement device waits for communication with the external terminal is saved, and thus, the power consumed for standby is saved. In addition, the user can easily grasp the period of time in which communication is executed in the biometric information measurement device. Thus, the user can easily recognize the timing to arrange the external terminal.

In the biometric information measurement device according to the above-described aspect, the light projector may visualize information related to measurement by irradiating the placement surface with light.

Here, the information related to measurement includes measurement information and measurement guidance information. According to this configuration, a configuration in which a display for displaying information related to measurement is not provided can be realized. Thus, the configuration of the biometric information measurement device can be simplified. Thus, the manufacturing cost of the biometric information measurement device can be reduced.

In addition, a terminal according to one aspect of the present invention includes: a receiver configured to receive measurement information from a biometric information measurement device by ultrasonic waves; and a display configured to display information that guides arrangement in a place to be arranged when information on the place to be arranged is visualized by the biometric information measurement device and the measurement information received by the receiver when the arrangement is performed according to the information that guides the arrangement in the place to be arranged.

According to this configuration, the user can easily place the terminal at a position with an appropriate relative relationship with respect to the biometric information measurement device. Thus, even in a case where the biometric information measurement device and the terminal communicate by ultrasonic waves having high directivity and a short reach distance, communication is easily established.

A biometric information measurement system may include: the biometric information measurement device according to the above-described aspect; and a terminal including a receiver configured to receive the information from the biometric information measurement device and a display configured to display the received information, wherein the display of the terminal is configured to display information that guides arrangement in a place to be arranged when information on the place to be arranged is visualized by the biometric information measurement device before the receiver receives the information, and the information that guides arrangement in the place to be arranged includes information indicating a direction in which the terminal is to be arranged in the place when the terminal is arranged in the place visualized by the light projector.

According to this configuration, before the terminal receives the information, the user can easily arrange the terminal in a direction in which ultrasonic waves easily reach, in addition to arranging the terminal at a place where the terminal is visualized by the light projector. Thus, power consumed for outputting ultrasonic waves in the biometric information measurement device is saved.

### Advantageous Effects of Invention

According to the present invention, it is possible to provide a technology for displaying an appropriate positional relationship between a biometric information measurement device and an external terminal in an easy-to-understand manner.

### BRIEF DESCRIPTION OF DRAWINGS

FIG. 1 schematically illustrates an example of an overview of a biometric information measurement system according to the present embodiment.

FIG. 2 schematically illustrates an example of an overview of a functional configuration of a blood pressure monitor.

FIG. 3 schematically illustrates an example of an overview of a functional configuration of a smartphone.

FIG. 4 schematically illustrates an example of a flowchart of processing performed by the blood pressure monitor.

FIG. 5 schematically illustrates an example of a flowchart of processing performed by a smartphone.

FIG. 6 illustrates an example of a flowchart of processing performed by the biometric information measurement system.

FIG. 7 schematically illustrates an example of an overview of a blood pressure monitor.

### DESCRIPTION OF EMBODIMENTS

An embodiment according to an aspect of the present invention (hereinafter, also referred to as “the present embodiment”) will be described below with reference to the drawings. However, the present embodiment described below is merely illustrative of the invention in all respects. Of course, various modifications and variations can be made without departing from the scope of the present invention. Thus, specific configurations in accordance with an embodiment may be adopted as appropriate at the time of carrying out the present invention.

### § 1 Application Example

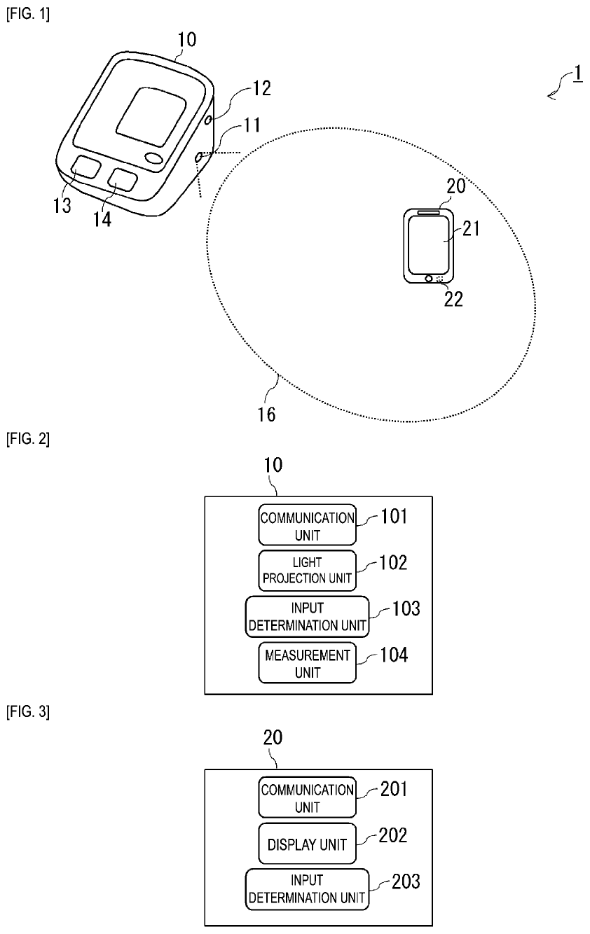

FIG. 1 schematically illustrates an example of an overview of a biometric information measurement system 1 according to the present embodiment. As illustrated in FIG. 1, the biometric information measurement system 1 includes a blood pressure monitor 10 for measuring blood pressure, which is an example of biometric information, and a smartphone 20.

The blood pressure monitor 10 includes an LED 11 provided on the side surface of the body so as to irradiate the upper surface of a table with light, for example. The blood pressure monitor 10 includes an ultrasonic module 12 that emits ultrasonic waves. A range 16 irradiated by the LED 11 is a recommended arrangement position of the smartphone 20 when communicating with the blood pressure monitor 10 via ultrasonic waves. Here, in the case of ultrasonic communication from the blood pressure monitor 10, unlike bidirectional communication such as Bluetooth (trade name), it is not possible to know in advance that communication is possible, so it is desirable to keep the arrangement relationship of the blood pressure monitor 10 and the smartphone 20 in an appropriate manner in order to establish communication.

According to the biometric information measurement system 1 as described above, the recommended arrangement position of the smartphone 20 is visualized by the irradiation of the LED 11. Therefore, the user can easily understand the place where the smartphone 20 is arranged. Therefore, the user can easily place the smartphone 20 with respect to the blood pressure monitor 10 in the range irradiated by the LED 11. Therefore, even when the blood pressure monitor 10 and the smartphone 20 communicate with each other by ultrasonic waves having high directivity and a short reach distance as in the biometric information measurement system 1 as described above, the establishment of communication can be easily achieved.

### § 2 Configuration Example

### System Configuration

As illustrated in FIG. 1, the biometric information measurement system 1 includes the blood pressure monitor 10 for measuring blood pressure, which is an example of biometric information, and the smartphone 20. Here, the blood pressure monitor 10 is an example of the “biometric information measurement device” of the present invention. The smartphone 20 is an example of the “terminal” of the present invention. The blood pressure monitor 10 and the smartphone 20 are placed on the same plane such as the upper surface of a table.

The blood pressure monitor 10 includes the LED 11 provided on the side surface of the body so as to irradiate the upper surface of a table with light, for example. The blood pressure monitor 10 includes the ultrasonic module 12 that emits ultrasonic waves. The blood pressure monitor 10 includes, on the front surface, a measurement button 13 for starting measurement of blood pressure, and a communication button 14 for starting communicating the measured blood pressure information to an external terminal. The range 16 irradiated by the LED 11 is the recommended arrangement position of the smartphone 20 when communicating with the blood pressure monitor 10 via ultrasonic waves. Here, the LED 11 is an example of the “light projector” of the present invention. The ultrasonic module 12 is an example of the “communicator” of the present invention. The range 16 irradiated by the LED 11 is an example of the “information on the place where the external terminal should be arranged” of the present invention. The blood pressure monitor 10 is provided with a cuff (not illustrated) that is wrapped around an arm during measurement.

On the other hand, the smartphone 20 includes a touch panel display 21 for displaying and inputting information, and a microphone 22 for detecting ultrasonic waves.

In the biometric information measurement system 1 according to the present embodiment, the blood pressure monitor 10 and the smartphone 20 communicate with each other by ultrasonic waves.

### Functional Configuration

FIG. 2 schematically illustrates an example of an overview of the functional configuration of the blood pressure monitor 10. The blood pressure monitor 10 includes a communication unit 101. The communication unit 101 is formed including the ultrasonic module 12. The communication unit 101 transmits information to an external terminal via ultrasonic waves. The communication unit 101 allows the ultrasonic module 12 to be able to communicate with an external terminal, and determines whether a predetermined period of time has elapsed in the communicable state.

The blood pressure monitor 10 includes a light projection unit 102. The light projection unit 102 is formed including the LED 11. The light projection unit 102 irradiates the recommended arrangement place with light when communicating with the blood pressure monitor 10 via ultrasonic waves. The light projection unit 102 adjusts the turning on and off of the LED 11 by controlling the current flowing into the LED 11.

The blood pressure monitor 10 includes an input determination unit 103. The input determination unit 103 determines whether the measurement button 13 and the communication button 14 are pressed.

The blood pressure monitor 10 includes a measurement unit 104. The measurement unit 104 measures blood pressure using a known measurement means.

FIG. 3 schematically illustrates an example of an overview of the functional configuration of the smartphone 20. The smartphone 20 is a portable terminal including a processor such as a CPU, a main storage device such as a RAM or ROM, a hard disk drive, an auxiliary storage device such as a removable medium, the touch panel display 21 having an input function and a display function, and the microphone 22 for detecting ultrasonic waves. Here, the smartphone 20 may include a microphone for detecting audible sound, and the microphone may also serve to detect ultrasonic waves. An operating system (OS), various programs, various tables, and the like are stored in the auxiliary storage device, and the programs stored therein are loaded into a work area of the main storage device and executed, and each component and the like are controlled by execution of the programs. In this way, each function that meets a predetermined purpose as described later can be realized.

The smartphone 20 includes the communication unit 201. The communication unit 201 is formed including the microphone 22. The communication unit 201 determines whether information has been received from an external device via the microphone 22 and performs the process of receiving the information. The communication unit 201 puts the microphone 22 into a standby state of waiting for communication with an external device, and determines whether a predetermined period of time has elapsed in the standby state.

The smartphone 20 includes the display unit 202. The display unit 202 is formed including the touch panel display 21. The display unit 202 displays the information on the touch panel display 21.

The smartphone 20 includes the input determination unit 203. The input determination unit 203 determines whether the information has been input via the touch panel display 21.

### § 3 Operation Example

### Processing Flow of Blood Pressure Monitor 10

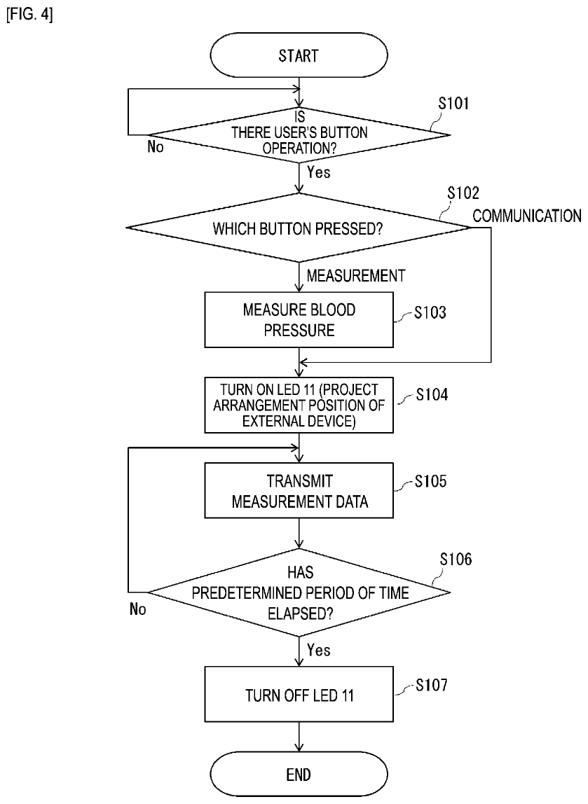

Next, the overview of the process executed by the blood pressure monitor 10 will be described. FIG. 4 schematically illustrates an example of a flowchart of processing executed by the blood pressure monitor 10.

### S101

In step S101, the input determination unit 103 determines whether there is a button operation from the user.

### S102

In step S102, when it is determined that there is a button operation, the input determination unit 103 determines whether the measurement button 13 or the communication button 14 has been pressed. When the input determination unit 103 determines that the measurement button 13 has been pressed, the process proceeds to step S103. On the other hand, when the input determination unit 103 determines that the communication button 14 has been pressed, the process proceeds to step S104.

### S103

In step S103, when it is determined that the measurement button 13 has been pressed, the measurement unit 104 measures blood pressure.

### S104

In step S104, when the measurement of blood pressure by the measurement unit 104 is completed, or when it is determined by the input determination unit 103 that the communication button 14 has been pressed, the light projection unit 102 turns on the LED 11.

### S105

In step S105, the communication unit 101 puts the ultrasonic module 12 into a transmittable state. The communication unit 101 transmits the measurement data to the smartphone 20. Here, the measurement data transmitted by the communication unit 101 is the data measured in step S103, but the measurement data may include information on the past measurement history measured by the measurement unit 104 and accumulated and stored in the blood pressure monitor 10.

### S1060

In step S106, the communication unit 101 determines whether a predetermined period of time has elapsed after the ultrasonic module 12 was put into the transmittable state. When it is determined that the predetermined period of time has elapsed, the process proceeds to step S107. On the other hand, when it is determined that the predetermined period of time has not elapsed, the process returns to step S105 and the process of transmitting the measurement data is executed again.

### S107

In step S107, the light projection unit 102 turns off the LED 11.

### Processing Flow of Smartphone 20

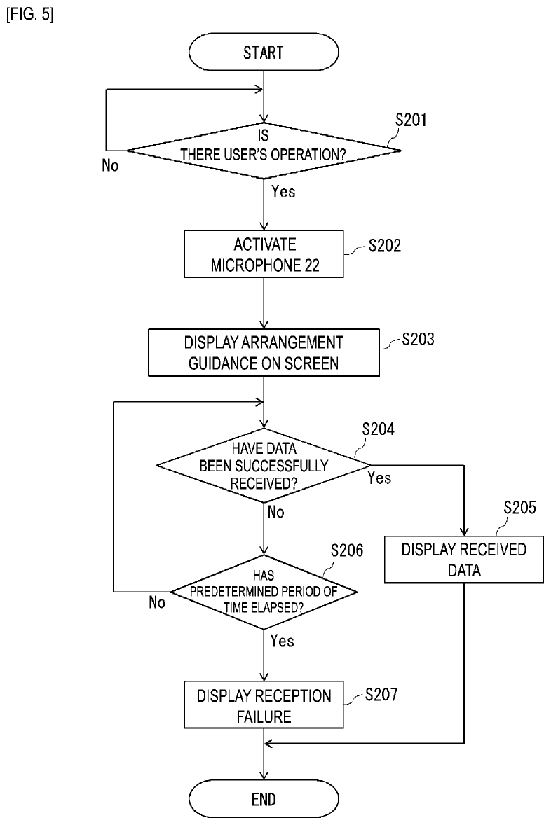

Next, the overview of the process executed by the smartphone 20 will be described. FIG. 5 schematically illustrates an example of a flowchart of processing executed by the smartphone 20.

### S201

In step S201, the input determination unit 203 determines whether there is an input operation from the user via the touch panel display 21. When it is determined that there has been an input operation, the process proceeds to step S202. In this case, the display unit 202 may display measurement guidance on the touch panel display 21 to wrap the cuff of the blood pressure monitor 10 and press the measurement button 13.

### S202

In step S202, the communication unit 201 activates the microphone 22 and puts it into a standby state of waiting for communication from the blood pressure monitor 10.

### S203

In step S203, the display unit 202 displays the arrangement guidance information on the touch panel display 21 to communicate with the blood pressure monitor 10. Here, the arrangement guidance information includes information for guiding the smartphone 20 to be placed within the range irradiated by the LED 11 of the blood pressure monitor 10. The arrangement guidance information includes information for displaying an arrow indicating the position of the microphone 22 (for example, an arrow indicating the lower part of the smartphone 20 when the microphone 22 is also used as a microphone for phone calls), and guiding the smartphone 20 to be placed in the direction in which the blood pressure monitor 10 comes in the direction of the arrow.

### S204

In step S204, the communication unit 201 determines whether the measurement data have been successfully received from the blood pressure monitor 10. When it is determined that the measurement data have been successfully received, the process proceeds to step S205. On the other hand, when it is determined that the measurement data have not been successfully received, the process proceeds to step S206.

### S205

In step S205, when it is determined that the measurement data can be received, the display unit 202 displays the received data on the touch panel display 21.

### S206

In step S206, when it is determined in step S204 that the measurement data have not been successfully received, the communication unit 201 determines whether a predetermined period of time has elapsed after the microphone 22 was put into the receivable state. When it is determined that the predetermined period of time has elapsed, the process proceeds to step S207. On the other hand, when it is determined that the predetermined period of time has not elapsed, the process returns to step S204, and the communication unit 201 determines again whether the measurement data has been received.

### S207

In step S207, the display unit 202 displays the reception failure information on the touch panel display 21 informing that reception of the measurement data from the blood pressure monitor 10 has failed.

### Processing Flow of Biometric Information Measurement System 1

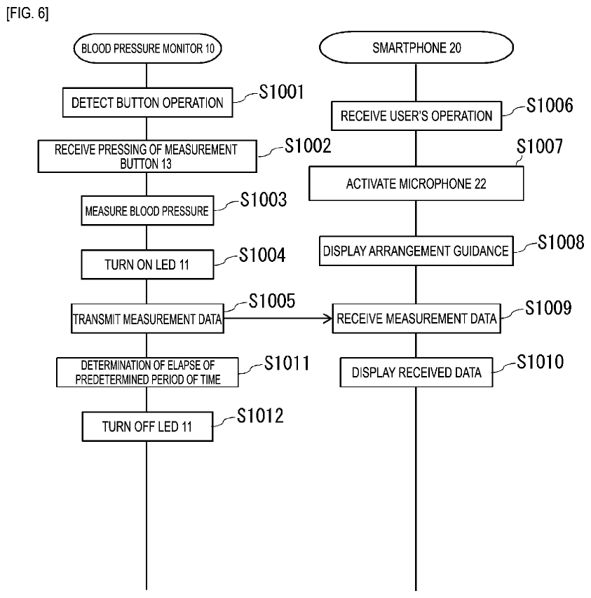

Next, the processing flow executed by the biometric information measurement system 1 will be described. FIG. 6 illustrates an example of a flowchart of processing executed by the biometric information measurement system 1.

### S1001

In step S1001, the input determination unit 103 of the blood pressure monitor 10 determines that the button has been operated by the user.

### S1002

In step S1002, the input determination unit 103 determines that the measurement button 13 has been pressed.

### S1003

In step S1003, the measurement unit 104 measures blood pressure.

### S1004

In step S1004, when the measurement of blood pressure by the measurement unit 104 is completed, the light projection unit 102 turns on the LED 11. Here, the light projection unit 102 controls the current flowing into the LED 11 so that the table portion in the range 16 on the right side from the portion where the blood pressure monitor 10 is placed is irradiated as illustrated in FIG. 1. Here, the range 16 irradiated by the LED 11 from the blood pressure monitor 10 is the recommended arrangement position of the smartphone 20 when communicating with the blood pressure monitor 10 via ultrasonic waves. As for the width, the distance from the blood pressure monitor 10 to the right end of the range may be, for example, about 30 cm. Further, depending on the place where the LED 11 of the blood pressure monitor 10 is provided, the range 16 irradiated by the LED 11 may be a range including the blood pressure monitor 10.

### S1005

In step S1005, the communication unit 101 transmits the measurement data to the smartphone 20.

### S1006

On the other hand, in the smartphone 20, the input determination unit 203 receives an input operation from the user via the touch panel display 21 in step S1006 independently of the processes of steps S1001 to S1005. The input operation is an operation related to receiving measurement data from the blood pressure monitor 10.

### S1007

In step S1007, the communication unit 201 activates the microphone 22 and puts it into a standby state of waiting for communication from the blood pressure monitor 10.

### S1008

In step S1008, the display unit 202 displays the arrangement guidance information on the touch panel display 21 to communicate with the blood pressure monitor 10. The guidance information includes, for example, instructing the smartphone 20 to be placed in the predetermined range 16 irradiated with light on the same table surface as the blood pressure monitor 10. The guidance information includes information for displaying an arrow indicating the position of the microphone 22 (for example, an arrow indicating the lower part of the smartphone 20 when the microphone 22 is also used as a microphone for phone calls), and guiding the smartphone 20 to be placed in the direction in which the blood pressure monitor 10 comes in the direction of the arrow. The user places the smartphone 20 in the predetermined range 16 on the table irradiated by the LED 11 of the blood pressure monitor 10 according to the guidance display.

### S1009

In step S1009, the communication unit 201 receives the measurement data from the blood pressure monitor 10.

### S1010

In step S1010, the display unit 202 displays the received data on the touch panel display 21.

### S1011

On the other hand, in the blood pressure monitor 10, in step S1011, the communication unit 101 determines whether a predetermined period of time has elapsed after the ultrasonic module 12 was put into the transmittable state. When it is determined that the predetermined period of time has elapsed, the process proceeds to step S1012.

### S1012

In step S1012, the light projection unit 102 turns off the LED 11.

### Actions and Effects

According to the biometric information measurement system 1 as described above, the recommended arrangement position of the smartphone 20 on the table on which the blood pressure monitor 10 is provided is visualized by the irradiation of the LED 11. Thus, the user can easily understand where to arrange the smartphone 20. Thus, the user can easily place the smartphone 20 with respect to the blood pressure monitor 10 in the range 16 irradiated by LED 11. Thus, according to the biometric information measurement system 1 as described above, even in a case where the blood pressure monitor 10 and the smartphone 20 communicate via ultrasonic waves having high directivity and a short reach distance, communication can be easily established.

According to the biometric information measurement system 1 as described above, the LED 11 is turned on in step S104 when the blood pressure monitor 10 starts transmitting the measurement data to the smartphone 20 as in a case where the blood pressure measurement is completed in step S103 or a case where it is determined that the communication button 14 has been pressed in step S102. Thus, when communication by the blood pressure monitor 10 is started, the user may be prompted to place the smartphone 20 into the range 16 irradiated by LED 11. Thus, according to the biometric information measurement system 1 as described above, the period of time in which the communication unit 101 of the blood pressure monitor 10 waits for communication with the smartphone 20 is saved, and thus, the power consumed for standby is saved. According to the biometric information measurement system 1 as described above, the user can easily understand when the communication unit 101 of the blood pressure monitor 10 is communicating. Thus, the user can easily recognize the timing to arrange the smartphone 20.

According to the biometric information measurement system 1 as described above, the user can easily understand a recommended arrangement direction of the smartphone 20 by looking at the guidance information on the direction of arrangement of the smartphone 20 displayed on the touch panel display 21 of the smartphone 20 in addition to arranging the smartphone 20 in the recommended place. The user may also place the smartphone 20 on a table so that the microphone 22 of the smartphone 20 is directed toward the ultrasonic module 12 of the blood pressure monitor 10 in accordance with the guidance information so that the establishment of communication between the blood pressure monitor 10 and the smartphone 20 is further facilitated.

### § 4. Modified Examples

4.1

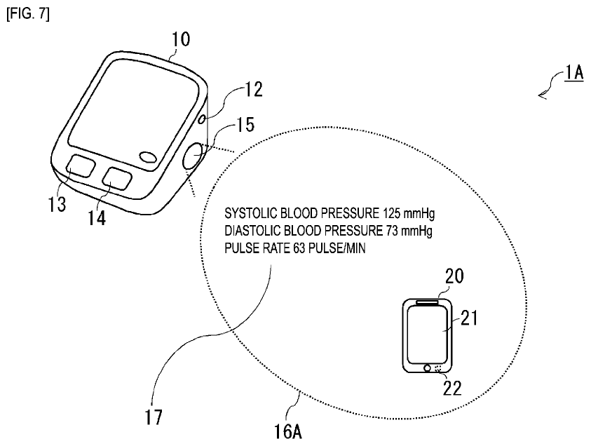

FIG. 7 schematically illustrates an example of the overview of a blood pressure monitor 10A according to the modified example. As illustrated in FIG. 7, the blood pressure monitor 10A includes a projector 15 instead of the LED 11. A light projection unit 102A is formed including the projector 15. The light projection unit 102A projects a graphic 16A of a recommended arrangement range of the smartphone 20 on the table by using the projector 15 when communication is performed by ultrasonic waves. The light projection unit 102A also projects information on measurement result 17 measured by the measurement unit 104 on the table. The light projection unit 102A may also project the measurement guidance information on the table. Here, the measurement result 17 and the measurement guidance information are examples of the “information related to measurement” of the present invention.

### Actions and Effects

According to the blood pressure monitor 10A as described above, a configuration in which a display for displaying the measurement result 17 is not provided in the blood pressure monitor 10A can be realized. Therefore, the configuration of the blood pressure monitor 10A can be simplified. Therefore, the manufacturing cost of the blood pressure monitor 10A can be reduced.

### Other Modified Examples

In the biometric information measurement system as described above, the LED 11 is turned on in step S104 when the blood pressure monitor 10 starts transmitting the measurement data to the smartphone 20 as in a case where the blood pressure measurement is completed in step S103 or a case where it is determined that the communication button 14 has been pressed in step S102. However, the timing at which the LED 11 is turned on is not limited to the example illustrated in the biometric information measurement system 1 described above. For example, the LED 11 may be turned on before the blood pressure monitor 10 starts transmitting measurement data to the smartphone 20. Additionally, the LED 11 may be turned off while the blood pressure monitor 10 is transmitting measurement data to the smartphone 20.

In the biometric information measurement system described above, an example in which the blood pressure monitor 10 and the smartphone 20 are placed on the same table surface is illustrated, but the blood pressure monitor 10 and the smartphone 20 may not be placed on the same table surface. The range 16 irradiated by the LED 11 of the blood pressure monitor 10 may not be formed on the surface on which the blood pressure monitor 10 is provided.

The light projector is not limited to the LED 11, and a laser irradiation module or the like may be provided in the blood pressure monitor 10, and the recommended arrangement place of the smartphone 20 may be visualized by the laser irradiation module or the like.

The embodiments disclosed above can be combined with each other.

In addition, in order to allow a comparison of the constituent features of the present invention with the configuration in the embodiments, the constituent features of the present invention will be described using the reference signs in the drawings.

### Supplementary Note 1

A biometric information measurement device (10, 10A) configured to communicate with an external terminal by ultrasonic waves, the biometric information measurement device (10, 10A) including:

a communicator (12) configured to communicate information including measurement information with the external terminal; and
a light projector (11) configured to visualize information (16) on a place where the external terminal is to be placed by irradiating light.

### Supplementary Note 2

The biometric information measurement device (10, 10A) according to Supplementary Note 1, wherein the place irradiated with light by the light projector (11) includes a place on a placement surface on which the host device is placed.

### Supplementary Note 3

The biometric information measurement device (10, 10A) according to Supplementary Note 1 or 2, wherein a period of time in which light is irradiated by the light projector (11) includes at least one of a predetermined period of time before execution of communication with the external terminal by the communicator (12) or at least a part of period of time in which the communication is executed.

### Supplementary Note 4

The biometric information measurement device (10, 10A) according to any one of Supplementary Notes 1 to 3, wherein the light projector (11) is configured to visualize information related to measurement by irradiating the placement surface with light.

### Supplementary Note 5

A terminal (20) including:

a receiver (22) configured to receive measurement information from a biometric information measurement device (10, 10A) by ultrasonic waves; and
a display (21) configured to display information that guides arrangement in a place to be arranged when information on the place to be arranged is visualized by the biometric information measurement device (10, 10A) and the measurement information received by the receiver (22) when the arrangement is performed according to the information that guides the arrangement in the place to be arranged.

### Supplementary Note 6

A biometric information measurement system (1, 1A) including:

the biometric information measurement device (10, 10A) according to any one of Supplementary Notes 1 to 4; and
a terminal (20) including a receiver (22) configured to receive information from the biometric information measurement device (10, 10A) and a display (21) configured to display the received information, wherein
the display (21) of the terminal (20) is configured to display information that guides arrangement in a place to be arranged when information on the place to be arranged is visualized by the biometric information measurement device (10, 10A) before the receiver (22) receives the information, and
the information that guides arrangement in the place to be arranged includes information indicating a direction in which the terminal (20) is to be arranged in the place when the terminal (20) is arranged in the place visualized by the light projector (11).

### REFERENCE SIGNS LIST

1, 1A: Biometric information measurement system
10, 10A: Blood pressure monitor
11: LED
12: Ultrasonic module
13: Measurement button
14: Communication button
15: Projector
16: Range irradiated by LED 11
16A: Graphic irradiated by projector 15
17: Measurement result
20: Smartphone
21: Touch panel display
22: Microphone
101: Communication unit
102, 102A: Light projection unit
103: Input determination unit
104: Measurement unit
201: Communication unit
202: Display unit
203: Input determination unit

1. A biometric information measurement device configured to communicate with an external terminal by ultrasonic waves, the biometric information measurement device comprising:
a communicator configured to communicate information including measurement information with the external terminal; and
a light projector configured to visualize information on a place where the external terminal is to be placed by irradiating light.

2. The biometric information measurement device according to claim 1, wherein the place irradiated with light by the light projector includes a place on a placement surface on which the host device is placed.

3. The biometric information measurement device according to claim 1, wherein a period of time in which light is irradiated by the light projector includes at least one of a predetermined period of time before execution of communication with the external terminal by the communicator or at least a part of period of time in which the communication is executed.

4. The biometric information measurement device according to claim 1, wherein the light projector is configured to visualize information related to measurement by irradiating the placement surface with light.

5. A terminal comprising:
a receiver configured to receive measurement information from a biometric information measurement device by ultrasonic waves; and
a display configured to display information that guides arrangement in a place to be arranged when information on the place to be arranged is visualized by the biometric information measurement device and the measurement information received by the receiver when the arrangement is performed according to the information that guides the arrangement in the place to be arranged.

6. A biometric information measurement system comprising:
the biometric information measurement device according to claim 1; and
a terminal including a receiver configured to receive the information from the biometric information measurement device and a display configured to display the received information, wherein
the display of the terminal is configured to display information that guides arrangement in a place to be arranged when information on the place to be arranged is visualized by the biometric information measurement device before the receiver receives the information, and
the information that guides arrangement in the place to be arranged includes information indicating a direction in which the terminal is to be arranged in the place when the terminal is arranged in the place visualized by the light projector.
